(19) United States
(12) Patent Application Publication
(10) Pub. No.: US 2025/0220285 A1
(43) Pub. Date: Jul. 3, 2025

(54) CAMERA HEAD WITH ADJUSTABLE MULTI-CHANNEL PRISM FOR SPECTRAL IMAGING AND METHODS OF USING THE SAME

(71) Applicant: KARL STORZ Imaging, Inc., Goleta, CA (US)

(72) Inventor: Christopher Zimmer, Goleta, CA (US)

(73) Assignee: KARL STORZ Imaging, Inc., Goleta, CA (US)

(21) Appl. No.: 18/398,834

(22) Filed: Dec. 28, 2023

(51) Int. Cl. H04N 23/16; A61B 1/00; A61B 1/04; A61B 1/06; G02B 23/24; H04N 23/11; H04N 23/15; H04N 23/50; H04N 23/58

(52) U.S. Cl. CPC H04N 23/16; A61B 1/00186; A61B 1/043; A61B 1/0638; G02B 23/243; H04N 23/11; H04N 23/15; H04N 23/555; H04N 23/58

(57) ABSTRACT

Methods and systems are provided to enable multiple fluorophores to be imaged by moving a prism assembly relative to the optical axis of collected light. Methods and systems to account for the shift of the prism assembly relative to the image sensors are also provided herein, such as software-implemented feature detection and registration, as well as complementary motion of the image sensors relative to the prism assembly.

### BACKGROUND

During the course of a surgery or surgical procedure, one or more images of a surgical site may be captured. The imaging may enable a surgeon to view the surgical site and make determinations based on the imaging. For example, the surgeon may use images or a live feed of a surgical site in maneuvering a surgical tool or to make a visual diagnosis, take a biopsy, etc. Different imaging modalities may be used during the surgery or surgical procedure for different purposes, with each imaging modality providing a different view or a different set of information about the surgical site.

Camera heads, usually connected to imaging scopes such as endoscopes or exoscopes, can provide multi-channel fluorescence imaging (FI) techniques for medical images. These FI techniques may include the imaging of multiple different fluorophores (also referred to herein as fluorescent markers), such as Indocyanine Green (ICG) and Cy5.5. However, each fluorophore has different emission and excitation spectra, making it difficult for a single camera head to capture images for multiple different FI modes with multiple different fluorophores. Additionally, the spectra of different fluorophores may overlap, increasing the difficulty of adequately separating wavelengths for multiple FI modes, since emission spectra of a first fluorophore may overlap with excitation spectra of a second, different fluorophore. Such an overlap may make it unclear whether light collected by the camera head is associated with excitation light or emission light, negatively impacting the final image. This may also limit the number of FI modes available to the user of the camera head, leading to user dissatisfaction.

### SUMMARY

At least the shortcomings of systems discussed above can be addressed with the systems and methods disclosed herein. By providing a multi-channel prism that can be moved or rotated to vary the cutoff wavelength of a dichroic filter, different wavelengths of light can be directed to different image sensors to enable different imaging modalities of different fluorophores. For example, the prism may be oriented such that white light and part of the near infrared (NIR) spectrum travels through a first channel of the prism, while the remainder of the NIR spectrum travels through a second channel of the prism. The prism's cutoff wavelength, which determines which wavelengths are sent to which channels, can be adjusted based on the orientation of the prism.

Additionally, and due to the movement of the multi-channel prism, light exiting the prism and reaching the image sensor may be shifted relative to the center of the image sensor, resulting in a shifted or offset image. Embodiments of the present disclosure account for such shifting using feature detection and registration and/or complementary movement of the image sensors. For example, a processor may, upon executing instructions, use image processing to perform feature detection across the image sensor channels and register the first image from the first image sensor with the second image from the second image sensor, such that the two can be overlaid and a composite, centered image can be rendered for a user to view. Additionally or alternatively, the image sensors themselves may be moved relative to the multi-channel prism to center the image sensors relative to light transmitted from the multi-dimensional prism. In such examples, the image sensors may be positioned on stages or platforms that are movable relative to the multi-channel prism, such that each sensor can be aligned with the multi-channel prism once the multi-channel prism has moved relative to the optical axis of the input light.

### BRIEF DESCRIPTION OF THE DRAWINGS

FIG. 1 shows a block diagram of a system according to at least one exemplary embodiment;

FIG. 2 shows a light source according to at least one exemplary embodiment;

FIG. 3A shows aspects of a camera head with a prism assembly in a first orientation according to at least one exemplary embodiment;

FIG. 3B shows aspects of the camera head with the prism assembly in a second orientation according to at least one exemplary embodiment;

FIG. 3C shows a chart of wavelength transmissions for a dichroic filter in different orientations according to at least one exemplary embodiment;

FIG. 3D shows aspects of another camera head with another prism assembly in a first orientation according to at least one exemplary embodiment;

FIG. 3E shows aspects of the other camera head with the other prism assembly in a second orientation according to at least one exemplary embodiment;

FIG. 4 shows an example of the emission curves of two fluorophores and the transmissions for a dichroic filter in different orientations according to at least one exemplary embodiment;

FIG. 5 shows aspects of centering channels of two image sensors according to at least one exemplary embodiment; and

FIG. 6 shows a method according to at least one exemplary embodiment.

### DETAILED DESCRIPTION

The exemplary systems and methods of this disclosure will be described in relation to imaging. However, to avoid unnecessarily obscuring the present disclosure, the description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

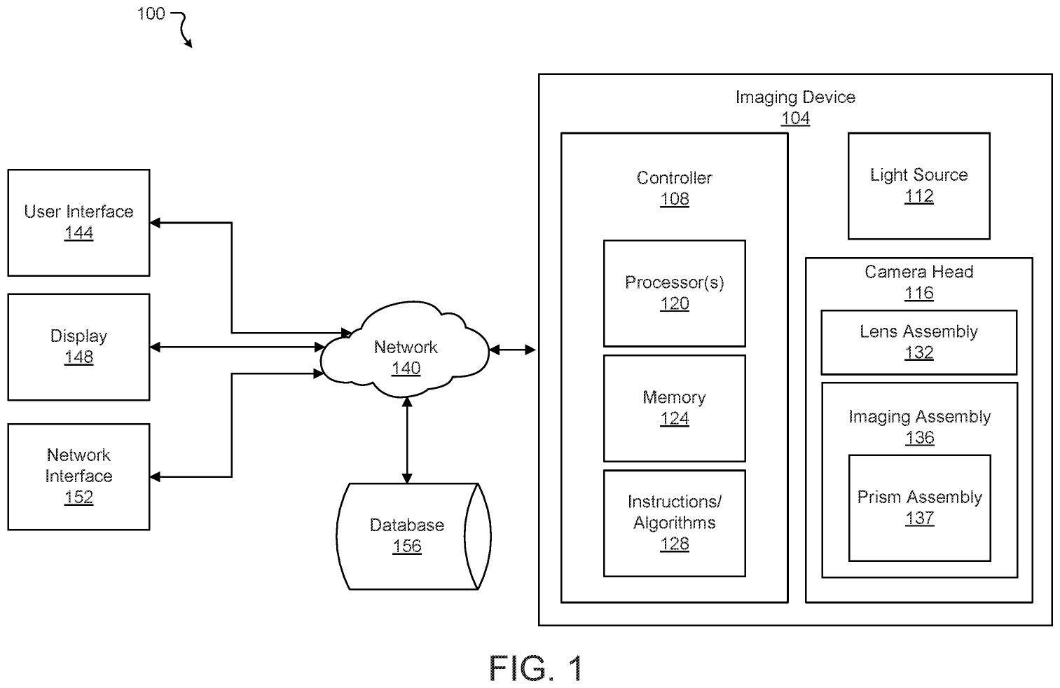

Turning first to FIG. 1, aspects of a system 100 are shown according to at least one exemplary embodiment. The system 100 includes an imaging device 104, a controller 108, a network 140, a user interface 144, a display 148, a network interface 152, and a database 156. In some embodiments, the system 100 may include additional or alternative components, and the components illustrated in FIG. 1 are in no way limiting. For example, the system 100 may include additional or alternative imaging devices, additional surgical tools (e.g., surgical drills, endoscopes, etc.), combinations thereof, and the like. As another example, the controller 108 provided in the imaging device 104 may alternatively be positioned outside of the imaging device 104 and may communicate with the imaging device 104 (or components thereof) wirelessly and/or through a wired connection. Such an external controller may be comprised in a camera control unit (CCU).

The imaging device 104 may be capable of capturing one or more images and/or video by illuminating a surgical scene with, for example, the light source 112. The light source 112 may provide a continuous illumination that reflects, scatters, and/or causes fluorescent emission from elements of the surgical scene, and such light is captured by a camera head 116. Often between the camera head and the surgical scene will be an intermediate device (not shown), such as an endoscope, borescope (for industrial applications), or optical elements of an exoscope, which capture light from the scene and may condition and relay the captured light to the camera head 116. It should be noted that light collected from the imaging scene includes all light gathered by imaging optics and passed into the camera head 116. Such light includes light reflected from elements in the scene, light scattered from surfaces, fluorescent light (e.g., excitation light) emitted from fluorophores, light directly coming from other illumination sources, combinations thereof, and/or the like.

The camera head 116 may be a device containing a lens assembly 132 and an imaging assembly 136 that measures light from an imaging scene such as a surgical scene. The imaging assembly 136 comprises a prism assembly 137 and two or more image sensors. The lens assembly 132 may include a plurality of optical components that condition, filter and/or direct light collected from the surgical scene into the prism assembly 137. The two or more image sensors of the imaging assembly 136 measure the collected light, with these measurements used by the controller 108 to generate one or more images of the surgical scene. While some embodiments discussed herein reference two image sensors, it is to be understood that other embodiments may comprise one or more additional image sensors dedicated to other imaging modalities not discussed herein, and that the number of image sensors present or available is in no way limited.

The processor 120 may provide processing functionality and may correspond to one or many computer processing devices. For instance, the processor 120 may be provided as a Field Programmable Gate Array (FPGA), an Application-Specific Integrated Circuit (ASIC), any other type of Integrated Circuit (IC) chip, a collection of IC chips, a microcontroller, a collection of microcontrollers, a GPU(s), or the like. As another example, the processor 120 may be provided as a microprocessor, Central Processing Unit (CPU), Graphics Processing Unit (GPU), and/or plurality of microprocessors that are configured to execute the instructions or algorithms 128 and/or data stored in memory 124. The processor 120 enables various functions of the system 100 upon executing the instructions or algorithms 128 and/or data stored in the memory 124.

The memory 124 may be or comprise a computer readable medium including instructions that are executable by the controller 108 and/or the processor 120. The memory 124 may include any type of computer memory device and may be volatile or non-volatile in nature. In some embodiments, the memory 124 may include a plurality of different memory devices. Non-limiting examples of memory 124 include Random Access Memory (RAM), Read Only Memory (ROM), flash memory, Electronically-Erasable Programmable ROM (EEPROM), Dynamic RAM (DRAM), etc. The memory 124 may include instructions (e.g., the instructions 128) that enable the controller 108 to control the various elements of the system 100 and to store data, for example, into the database 156 and retrieve information from the database 156. The memory 124 may be local (e.g., integrated with) the imaging device 104 or separate from the imaging device 104.

The instructions and/or algorithms 128 comprise computer-readable software that is executable by the controller 108 and/or the processor 120 that cause the controller 108 and/or the processor 120 to perform one or more functions. The instructions and/or algorithms 128 may, when processed by the processor 120, cause the processor 120 to perform one or more algorithms for image processing, for controlling one or more components of the system 100, combinations thereof, and the like. As an example, the instructions and/or algorithms 128 may cause the processor to perform one or more image processing techniques (e.g., edge detection techniques, interpolations, demosaicing algorithms, Bayer filter pattern algorithms, image overlays, etc.) to convert measurements from the one or more image sensors into one or more images for storage and/or display.

The user interface 144 includes hardware and/or software that enables user input to the system 100 and/or any one or more components thereof. The user interface 144 may include a keyboard, a mouse, a touch-sensitive pad, touch-sensitive buttons, mechanical buttons, switches, and/or other control elements for providing user input to the system 100 to enable user control over certain functions of the system 100 (e.g., operating lighting and/or imaging capabilities of the imaging device 104, rendering processed video and/or images to the display 148, etc.). The user interface 144 may include buttons, switches, or other control means disposed on the imaging device 104 itself that are independent of or in addition to user interface controls not positioned in the imaging device 104. Simply as an illustrative example, the imaging device 104 and/or the display 148 may have input buttons and switches, and, additionally, a keyboard or mouse may be connected directly to the processor 120 (in embodiments where the processor 120 is located outside of the imaging device 104). All of these together constitute the user interface 144.

The display 148 may be or comprise a liquid crystal display (LCD), a light emitting diode (LED) display, a high definition (HD) display, a 4K display, or the like. The display 148 may be a stand-alone display or a display integrated as part of another device, such as a smart phone, a laptop, a tablet, a headset or head-worn device, and/or the like. In one embodiment, the display 148 may be a monitor or other viewing equipment included within an operating room, such that video feed captured from a surgery or surgical procedure can be rendered to the display 148 for a physician to view. In some embodiments, the display 148 may comprise a plurality of displays according to, for example, system design.

The network interface 152 may enable one or more components of the system 100 to communicate wired and/or wirelessly with one another or with components outside the system 100 via the network 140. The network interface 152 comprises one or more communication interfaces that permit the components of the system 100 to communicate using the network interface 152 and that include wired and/or wireless communication interfaces for exchanging data and control signals between one another. Examples of wired communication interfaces/connections include Ethernet connections, HDMI connections, connections that adhere to PCI/PCIe standards and SATA standards, and/or the like. Examples of wireless interfaces/connections include Wi-Fi connections, LTE connections, Bluetooth® connections, NFC connections, and/or the like.

The database 156 includes the same or similar structure as the memory 124 described above. In at least one exemplary embodiment, the database 156 is included in a remote server and stores video data captured during a surgery or surgical procedure (e.g., a camera on an endoscope capturing a live feed during an endoscopy).

Although FIG. 1 illustrates the various elements in the system 100 as being separate from one another, it should be appreciated that some or all of the elements may be integrated with each other if desired. For example, a single desktop or laptop computer or a CCU may include the processor 120, the memory 124, the user interface 144, and, optionally, the display 148. It should be further appreciated that each element in the system 100 includes one or more communication interfaces that enable communication with other elements in the system 100 over, for example, the network interface 152. In another example, an endoscope is combined with the camera head 116 (or detachably connected thereto) with a built in user interface 144 that is connected to an external camera control unit (CCU), the CCU comprising the controller 108, the memory 124, the processor 120, the network interface 152, and a user interface 144, and the CCU is also connected such that it can output image data to the display 148.

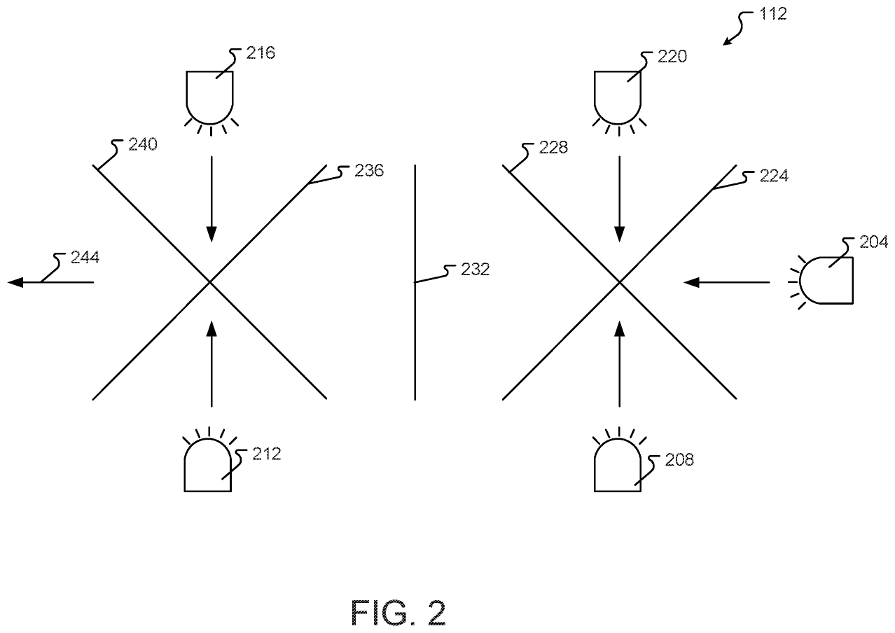

FIG. 2 illustrates aspects of an example light source 112 according to at least one exemplary embodiment of the present disclosure. The light source 112 may be or comprise an illumination device that includes a first illuminant 204, a second illuminant 208, a third illuminant 212, a fourth illuminant 216, and a fifth illuminant 220. Each of the illuminants may be capable of generating and/or emitting light at a set wavelength or a within a range of wavelengths. For example, each of the first illuminant 204, the second illuminant 208, the third illuminant 212, the fourth illuminant 216 and the fifth illuminant 220 may be or comprise LEDs. In one embodiment, the first illuminant 204 may generate green light (e.g., light with a central or most prevalent wavelength of about 550 nanometers (nm)) or, in some embodiments where a white light source is used, white light (e.g., light with wavelengths between 400 nm and 700 nm); the second illuminant 208 may generate blue light (e.g., light with a central or most prevalent wavelength of about 450 nm); the third illuminant 212 may generate infrared (IR) light with a wavelength of about 940 nm; the fourth illuminant 216 may generate infrared light with a wavelength of about 770 nm; and the fifth illuminant 220 may generate red light with a central or most prevalent wavelength of about 630 nm.

The light generated by the illuminants may pass through a series of filters and/or beam combiners before being output from the light source 112, such that the final light output by the light source 112 includes a spectrum of wavelengths generated by the separate illuminants. For example, a first filter/beam combiner 224 may reflect red light and transmit all other light, such that the red light generated by the fifth illuminant 220 is directed toward a third filter 232. Similarly, a second filter/beam combiner 228 may reflect light with wavelengths below 500 nm and transmit light with wavelengths above 500 nm. As a result, the second filter/beam combiner 228 may direct the blue light generated by the second illuminant 208 toward the third filter 232. The filtering parameters of the first filter/beam combiner 224 and the second filter/beam combiner 228 may enable the green light generated by the first illuminant 204 to pass through both the first filter/beam combiner 224 and the second filter/beam combiner 228 as the green light travels toward the third filter 232. This configuration of filters and illuminants allows for the precise selection of spectral bands (also referred to herein as a set or range of wavelengths) in the combined light output 244. Adjustment of the spectral values (e.g., wavelengths of light) of the illuminants and the filters of the light source can be done in order to correspond to the desired configuration of the imaging assembly 136 of the camera head 116, as discussed in further detail below.

The third filter 232 may operate to separate the visible light illuminants from the infrared illuminants by permitting light with wavelengths below 650 nm to pass therethrough, but reflecting light with wavelengths above 650 nm. As a result, the visible light generated and emitted by the first illuminant 204, the second illuminant 208, and/or the fifth illuminant 220 pass through the third filter 232, while any infrared light generated by the first illuminant 204, the second illuminant 208, and/or the fifth illuminant 220 is reflected. The placement of the third filter 232 may ensure that no infrared light that may be output from the first illuminant 204, the second illuminant 208, and/or the fifth illuminant 220 is an element of combined light output 244. A fourth filter/beam combiner 236 may reflect light with wavelengths between about 725 and 800 nm, while transmitting light of wavelengths shorter than 725 nm and greater than 800 nm. The fourth filter/beam combiner 236 directs the infrared light generated by the fourth illuminant 216 (which may be about 770 nm in wavelength) toward the exit of the light source 112. Similarly, a fifth filter/beam combiner 240 may reflect wavelengths of about 940 nm, while letting all other wavelengths pass therethrough; the fifth filter/beam combiner 240 ensures the infrared light generated by the third illuminant 212 (which may be about 940 nm in wavelength) is directed toward the exit of the light source 112. The fourth filter/beam combiner 236 and the fifth filter/beam combiner 240 permit visible wavelengths to pass therethrough and out of the light source 112. In some embodiments, filtration may not be critical in the light source, and therefore filter/beam combiners 224, 228, 236, 240 may act only as beam combiners and the third filter 232 may not be present.

As shown in FIG. 2, the positioning of the first filter/beam combiner 224, the second filter/beam combiner 228, the third filter 232, the fourth filter/beam combiner 236, and the fifth filter/beam combiner 240 results in a combined light output 244 being output from the light source 112, with the combined light output 244 containing the wavelengths of light generated by each illuminant. In other words, the combined light output 244 may include two, three, or more spectrally distinct portions of light based on the number of illuminants active. As used herein, the term “spectrally distinct” means a different range, number, set, or content of wavelengths of light. In some cases, a first portion of light may overlap in spectral content with a second portion of light but may be considered spectrally distinct when the first portion of light contains spectral content (e.g., a set or range of wavelengths of light) that are not present in the second portion of light (or vice versa).

In some embodiments, the light source 112 may be strobed. In other words, the first illuminant 204, the second illuminant 208, the third illuminant 212, the fourth illuminant 216, and/or the fifth illuminant 220 may be modulated or duty cycled to periodically emit light, such that the combined light output 244 periodically illuminates the surgical site. In other embodiments, the light source 112 may not be strobed. In other words, the first illuminant 204, the second illuminant 208, the third illuminant 212, the fourth illuminant 216, and/or the fifth illuminant 220 may constantly generate light, such that the combined light output 244 illuminates the surgical site as long as the light source 112 remains on. It should also be noted that, while FIG. 2 shows a particular light source which may be employed with or as part of the present invention, other light sources which are known in the art and which produce the same or similar illumination characteristics may alternatively be employed.

As used herein and unless otherwise specified, the listing of wavelengths of light include approximate wavelength values. For example, green light with a wavelength of 550 nm may also include, for example, a percentage variation thereof (e.g., 1% below and above the 550 nm value, 2% below and above the 550 nm value, 10% below and above the 550 nm value, 25% below and above the 550 nm value, etc.). Furthermore, the term “about” includes the percentage variation of the wavelength of light. For example, “about 550 nm” may encompass all wavelengths between 544.5 and 555.5 nm. In some cases, the listed wavelength of light may be a wavelength at which a broader spectrum or range of wavelengths constituting the light is centered, and/or the most prevalent wavelength in the spectrum of light. As an example, the green light with a wavelength of 550 nm may indicate that the green light includes a broad spectrum (e.g., range) of wavelengths between, say, 485 nm and 615 nm that is centered at 550 nm, and/or that the 550 nm wavelength is the most prevalent or frequent wavelength in the spectrum.

As can be appreciated, such broad spectrums of wavelengths are not limited to green light, and other illumination sources discussed herein may similarly produce light with a broad spectrum of wavelengths. In some preferred embodiments, the light generated by different light sources may have overlapping spectra (e.g., ranges of wavelengths that overlap one another). In other words, the fifth illuminant 220 and the first illuminant 204 may both generate light that includes an overlapping range of wavelengths (e.g., overlapping wavelengths between 560 nm and 590 nm, overlapping wavelengths between 550 nm and 600 nm, etc.), and the first illuminant 204 and the second illuminant 208 may both generate light that includes an overlapping range of wavelengths (e.g., overlapping wavelengths between 480 nm and 550 nm, overlapping wavelengths between 450 nm and 580 nm, etc.). It is to be understood that these exemplary ranges, however, are in no way limiting, and broader or narrow ranges of spectral overlap of light generated by different illuminants are possible.

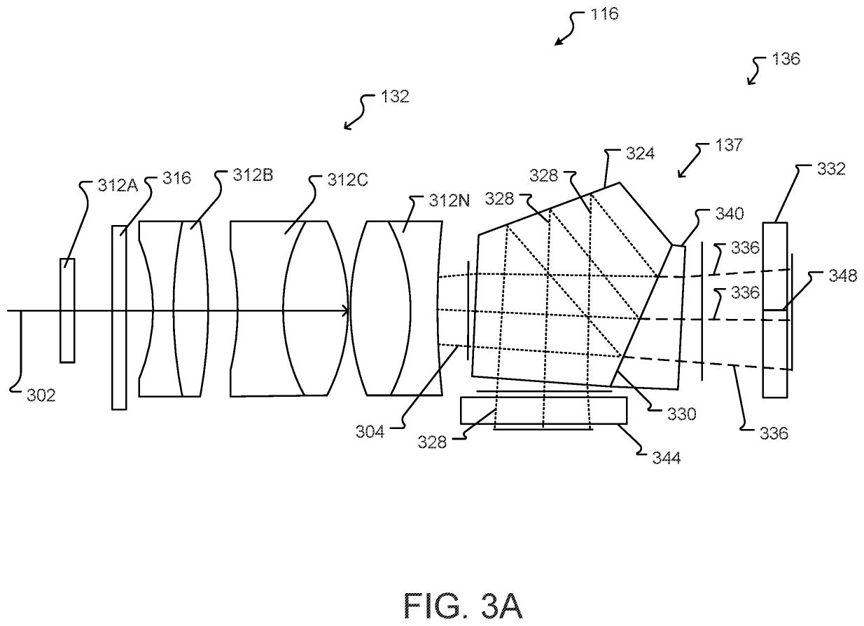

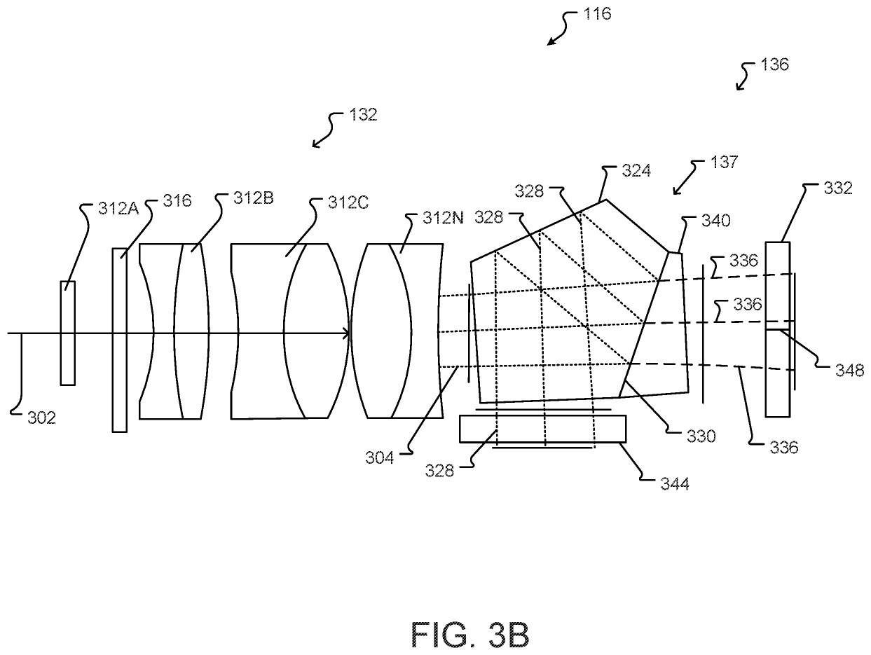

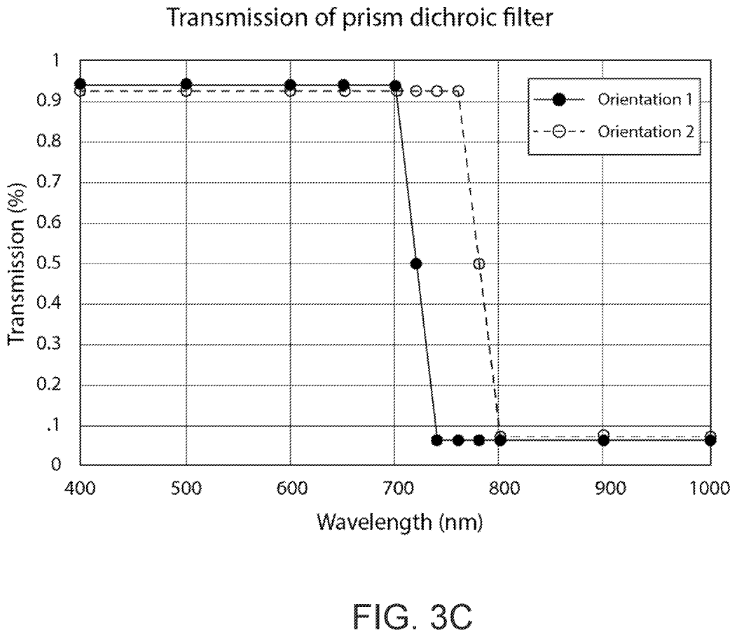

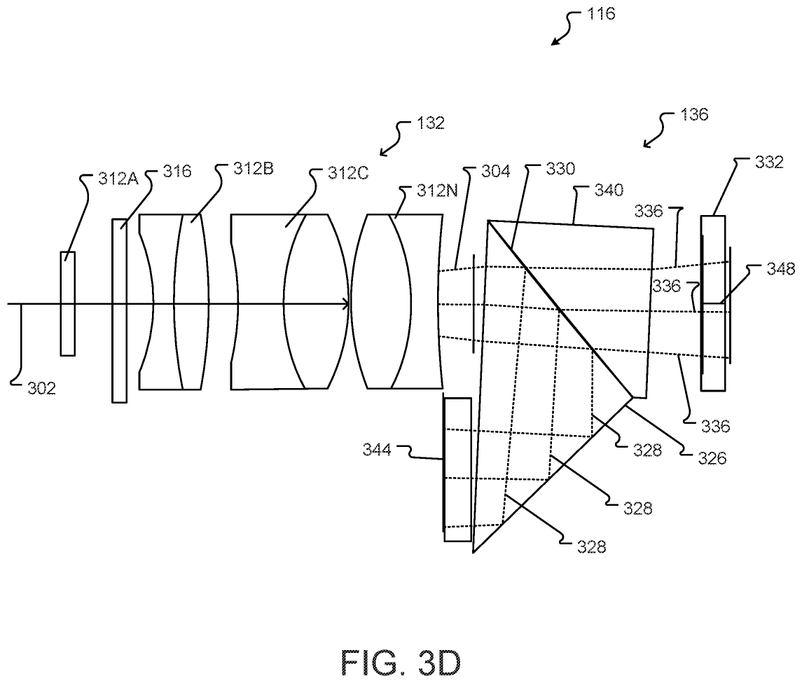

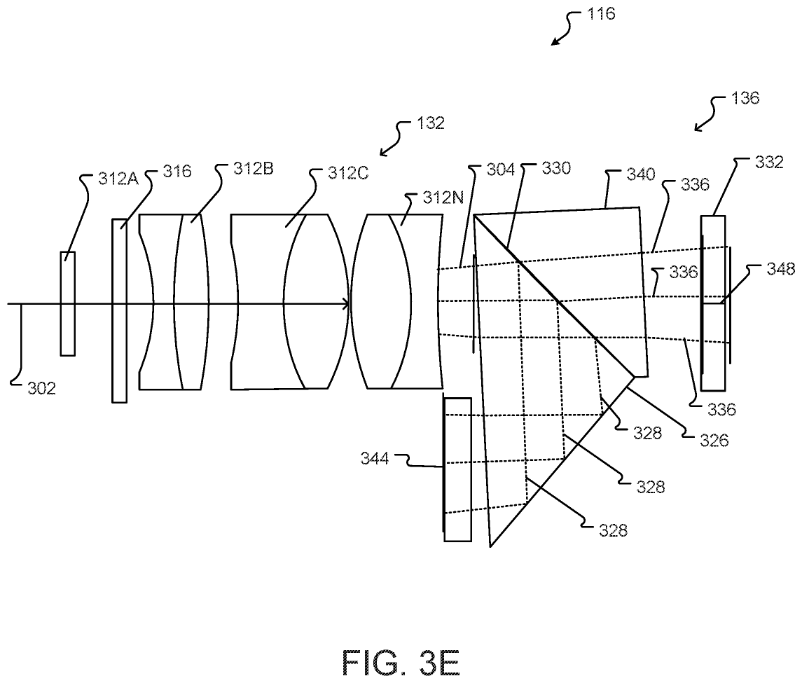

FIGS. 3A-3B illustrate aspects of a camera head 116 according to at least one exemplary embodiment of the present disclosure. The camera head 116 includes a lens assembly 132 and an imaging assembly 136 including a prism assembly 137. The lens assembly 132 may include a collection of mirrors, lenses, filters, polarizers, and/or windows capable of conditioning and directing collected light into the prism assembly 137. As shown in FIGS. 3A-3B, the lens assembly 132 includes a plurality of optical elements 312A-312N and a first filter 316, which may direct the light 302 to the prism assembly 137. In some embodiments, the light 302 may be light collected from a surgical scene (e.g., light reflected off the surgical scene, light from the light source 112, light emitted from one or more fluorophores, etc.) and captured by the optics of an attached endoscope or those of an exoscope and relayed or transmitted to the camera head 116.

The first filter 316 may filter out one or more wavelengths of the light captured by the camera head 116. The first filter 316 may be a replaceable, selectable, or tunable filter, such that its characteristics may be selected depending on the requirements of the desired mode. For example, in one embodiment the first filter 316 may be a dichroic filter that passes light in a first range of wavelengths but rejects light in a second range of wavelengths. In a further embodiment, the first filter 316 may be a different dichroic filter that passes light from a third range of wavelengths and rejects light from a fourth range of wavelengths. Various particular implementations are discussed below. In one embodiment, the first filter 316 may operate to filter out or block excitation wavelengths associated with one or more fluorophores in the surgical scene. For example, ICG may be present and induced to fluoresce in response to an excitation light with a wavelength of about 789 nm, and the first filter 316 may be configured to filter out light with wavelengths around about 789 nm, such that the peak excitation wavelength associated with ICG does not reach the imaging assembly 136. It is to be understood that, while the first filter 316 is illustrated as being positioned just before the plurality of optical elements 312A-312N in the lens assembly 132, in some cases the first filter 316 may be external to the camera head 116, such as when the first filter 316 is an element of an attached endoscope.

The lens assembly 132 may condition the light 302 such that conditioned light 304 passes into the prism assembly 137 and is ultimately focused onto a first image sensor 332 or a second image sensor 344, depending on the spectral content of the conditioned light 304. The prism assembly 137 includes a pentaprism 324 and a second prism 340 with a dichroic filter 330 positioned therebetween. In some alternative embodiments, the pentaprism 324 may be replaced by a right angle prism 326 as depicted in FIGS. 3D-3E. In such embodiments, the dichroic filter 330 may be disposed between the right angle prism 326 and the second prism 340, such that the prism assembly 137 operates in the same way with the right angle prism 326 as with the pentaprism 324.

The elements of the prism assembly 137 operate together as a multi-channel prism that receives the conditioned light 304 and separates the conditioned light 304 into at least two separate, spectrally distinct portions of light that are respectively directed to the first image sensor 332 and the second image sensor 344. The dichroic filter 330 located between the pentaprism 324 and the second prism 340 may be disposed on a surface of the pentaprism 324 or on a surface of the second prism 340. The dichroic filter 330 filters and/or reflects different wavelengths of light, creating a cutoff between different spectral bands depending on the angle of light incident on the dichroic filter 330. With reference to FIG. 3C that depicts transmission percentage of the dichroic filter 330 for a range of wavelengths, when incoming light hits the dichroic filter 330 at a first angle of incidence (such as when the prism assembly 137 is in a first orientation), light with wavelengths below about 720 nm is transmitted while light with wavelengths above about 720 nm is reflected. As another example, when incoming light hits the dichroic filter 330 at a second angle of incidence different from the first angle (such as when the prism assembly 137 is in a second orientation), light with wavelengths below about 780 nm is transmitted while light with wavelengths above about 780 nm is reflected. As a result, the spectral band of wavelengths transmitted by the dichroic filter 330 changes as a function of angle of incidence, such that movement of the prism assembly 137 enables a spectral shift of the band of wavelengths transmitted through the dichroic filter 330.

When the conditioned light 304 passes through the pentaprism 324 and reaches the dichroic filter 330, a portion of the conditioned light 304 is reflected as reflected light 328, while a portion of the conditioned light 304 is transmitted through the dichroic filter 330 as transmitted light 336. The spectral content of the reflected light 328 and the transmitted light 336 will vary depending upon the angle of incidence of the conditioned light 304 on the dichroic filter 330, which angle may be adjusted based on the orientation of the prism assembly 137, as discussed in further detail below. The reflected light 328 may be bent by or reflected inside the pentaprism 324 until the reflected light 328 passes into the second image sensor 344. The second image sensor 344 may receive the reflected light 328 and photosensitive elements (e.g., photodiodes, pixels) within the second image sensor 344 may generate corresponding electric signals. In some embodiments, the electric signals may be passed to one or more other components of the system 100 (such as to the controller 108) and further used to generate one or more images. The transmitted light 336 that has passed through the dichroic filter 330 is further transmitted by the second prism 340 into the first image sensor 332. The first image sensor 332 may receive the transmitted light 336 and photosensitive elements (e.g., photodiodes, pixels) within the first image sensor 332 may generate electric signals that can be used by the controller 108 to generate one or more images.

The prism assembly 137 may be positioned on a motorized movable stage or platform such that the prism assembly 137 can be moved between two or more discrete orientations to change the angle of incidence of the conditioned light 304 on the dichroic filter 330, and thus the spectral content of the reflected light 328 and the transmitted light 336. For example, the prism assembly 137 may be moved into a first orientation, such that the dichroic filter 330 has an effective cutoff wavelength of about 720 nm. In one embodiment, the first orientation comprises the prism assembly 137 being rotated several degrees clockwise from the optical axis of the conditioned light 304, resulting in a blueshift of the cutoff wavelength. In the first orientation, light below about 720 nm (e.g., light with wavelengths from about 400 nm to about 710 nm) is transmitted through the dichroic filter 330 as transmitted light 336 and passes into the first image sensor 332. Light above about 720 nm (e.g., light with wavelengths from about 730 nm to about 1000 nm) is reflected by the dichroic filter 330 and is eventually channeled by the pentaprism 324 into the second image sensor 344.

The prism assembly 137 may be moved by motors connected to the prism assembly 137. For example, the processor 120 may cause motors coupled with the prism assembly 137 to actuate, such that the prism assembly 137 moves on the movable stage or platform into the first orientation or the second orientation. In other examples, the prism assembly 137 may be manually moved by the user, such as by actuating a lever, button, or other mechanical device connected to the prism assembly 137 platform such that the prism assembly 137 moves into the first orientation or the second orientation. In yet another example, the platform on which the prism assembly 137 rests may be rotatable, such that electromechanical elements, stepper motors, rotating solenoids, other actuators, combinations thereof, and/or the like can be controlled by the processor 120 to rotate the prism assembly 137 into the first orientation or the second orientation.

When the prism assembly 137 is moved into a second orientation, the angle of incidence of the conditioned light 304 on the dichroic filter 330 changes. In one embodiment, the second orientation is a counterclockwise rotation of a few degrees from the prism assembly 137 being perpendicular with the optical axis of the conditioned light 304, causing a redshift such that the effective cutoff wavelength of the dichroic filter 330 is about 780 nm. In the second orientation, light below about 780 nm (e.g., light with wavelengths from about 400 nm to about 770 nm) is transmitted through the dichroic filter 330 as transmitted light 336, and passes into the first image sensor 332. Light above about 780 nm (e.g., light with wavelengths from about 790 nm to about 1000 nm) is reflected by the dichroic filter 330 and is eventually channeled by the pentaprism 324 into the second image sensor 344.

The difference in orientations of the prism assembly 137 may enable the camera head 116 to optimize image quality for any of multiple different fluorophores by changing the orientation of the prism assembly 137 by maximizing the amount of emission light passing to the respective FI sensor. For instance, ICG has an emission spectrum with a peak intensity around 830 nm, OTL38 has an emission spectrum with a peak intensity around 790 nm, and Cy7 has an emission spectrum with a peak intensity around 780 nm. Since all three of these fluorescent markers have emission spectrums with peak intensities above the 720 nm cutoff, the first orientation of the prism assembly 137 enables any one of these fluorophores to be imaged by the second image sensor 344. In other words, light associated with the emission of any of the above three fluorescent markers would be reflected by the dichroic filter 330 while the prism assembly 137 is in the first orientation and directed into the second image sensor 344, while the remaining light would be transmitted through the dichroic filter 330 to the first image sensor 332. As a result, the camera head 116 may be able to image a white light image on the first image sensor 332, while imaging a fluorescent image of, say, ICG (or OTL38 or Cy7) on the second image sensor 344.

The prism assembly 137 can then be moved to the second orientation to image a different set of fluorescent marker emissions. For example, Cy5 has an emission spectrum with a peak intensity around 670 nm, while Cy5.5 has an emission spectrum with a peak intensity around 700 nm. But both the Cy5 and Cy5.5 emission spectrums also include a large amount of emission intensity in the 750-770 nm range. As a result, if Cy5 or Cy5.5 were imaged with the prism assembly 137 in the first orientation, both the first image sensor 332 and the second image sensor 344 would measure emission spectra associated with the fluorophore, resulting in a lower signal to noise ratio of the emission signature of the fluorophore when the image from the first image sensor 332 and the image from the second image sensor 344 are overlaid. To address this issue, the prism assembly 137 may be moved to the second orientation, such that the dichroic filter 330 cutoff wavelength is about 780 nm. As a result, light with wavelengths from about 400 nm to about 770 nm (which may include emission spectra of Cy5 or Cy5.5) is transmitted through the dichroic filter 330 and directed to the first image sensor 332, while light with wavelengths from about 790 nm to about 1000 nm is reflected by the dichroic filter 330 and eventually channeled to the second image sensor 344. An example list of different fluorescent markers and the orientation of the prism assembly 137 is shown in Table 1 below.

TABLE 1
Prism Orientation for Various Fluorescent Markers

| | | Cutoff | Transmission | Reflection |
|---|---|---|---|---|
| Fluorescent | Prism | Wavelength | Region | Region |
| Marker | Orientation | (nm) | (nm) | (nm) |
| ICG | First | 720 | 400-710 | 730-1000 |
| OTL38 | First | 720 | 400-710 | 730-1000 |
| Cy7 | First | 720 | 400-710 | 730-1000 |
| Cy5 | Second | 780 | 400-770 | 790-1000 |
| Cy5.5 | Second | 780 | 400-770 | 790-1000 |

The prism assembly 137 may be moved between the first orientation (depicted in FIG. 3A with the pentaprism 324 and in FIG. 3D with the right angle prism 326) and the second orientation (depicted in FIG. 3B with the pentaprism 324 and in FIG. 3E with the right angle prism 326) depending on the type of fluorophore(s) being imaged, which may be determined based on user input, based on the processor 120 executing instructions 128, combinations thereof, and/or the like. For example, the prism assembly 137 may be in the first orientation, and the user may determine that an image depicting the emission of Cy5 is desired. The user may provide an input (e.g., via the user interface 144) to that effect, and the system 100 may determine that the prism assembly 137 should be moved into the second orientation. In some embodiments, the processor 120 may cause motors coupled with the prism assembly 137 to actuate, such that the prism assembly 137 moves on the movable stage or platform from the first orientation to the second orientation.

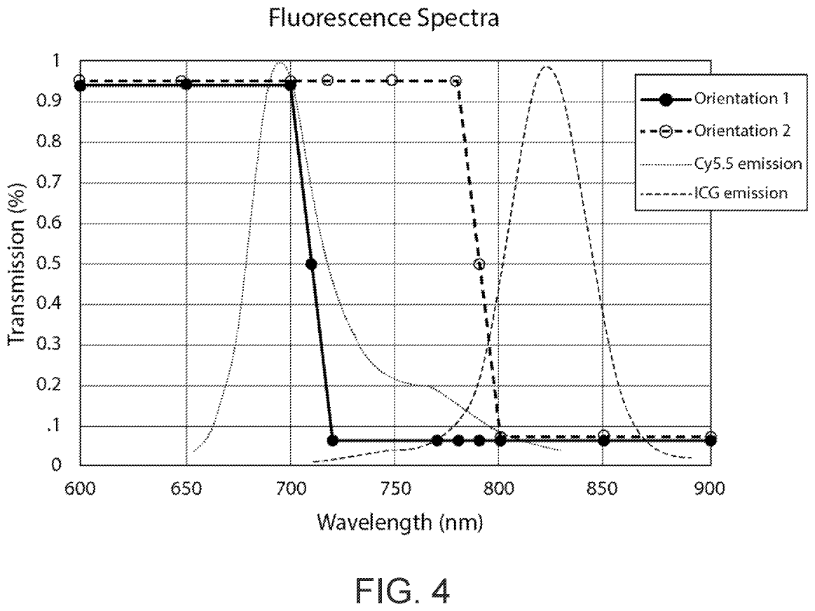

FIG. 4 shows an example of the transmission percentage of the dichroic filter 330 for a range of wavelengths when the prism assembly 137 is in a first orientation and a second orientation, particularly with reference to the emission spectra of two possible fluorophores, ICG and Cy5.5. As noted above, ICG has an emission maximum at about 830 nm, while Cy5.5 has an emission maximum around 700 nm. In a conventional system with only one cutoff wavelength, a user or manufacturer would have to decide on optimizing either for ICG or Cy5.5. This would result in more emission radiation for the non-optimized fluorophore being directed to a single sensor, decreasing thereby the signal-to-noise of the received signal. In the example shown, with the prism in a first orientation, the vast majority of ICG emission radiation will be sent to the second image sensor; however, if the prism was in the second orientation (that is, optimized for Cy5.5), one portion of the emission spectra would be sent to the first image sensor and another portion to the second image sensor. Thus, by selecting between two or more possible orientations of the prism assembly 137, embodiments of the system described herein allow for versatility in the optimization of signal-to-noise for a plurality of possible fluorophores.

In other embodiments, the prism assembly 137 may be automatically moved from the first orientation to the second orientation (or vice versa) when a surgical procedure proceeds to a predetermined surgical step. For instance, the surgical procedure may reach a step where ICG fluorescence is imaged, and the processor 120 may automatically drive the motors to move the stage or platform to which the prism assembly 137 is connected to move the prism assembly 137 into the first orientation. In some cases, the movement between orientations may be a fixed, known, and/or predetermined movement (e.g., a counterclockwise rotation of 5 degrees), with such information stored in the memory 124 and/or the database 156 and accessed by the processor 120 when controlling the motors to move the prism assembly 137.

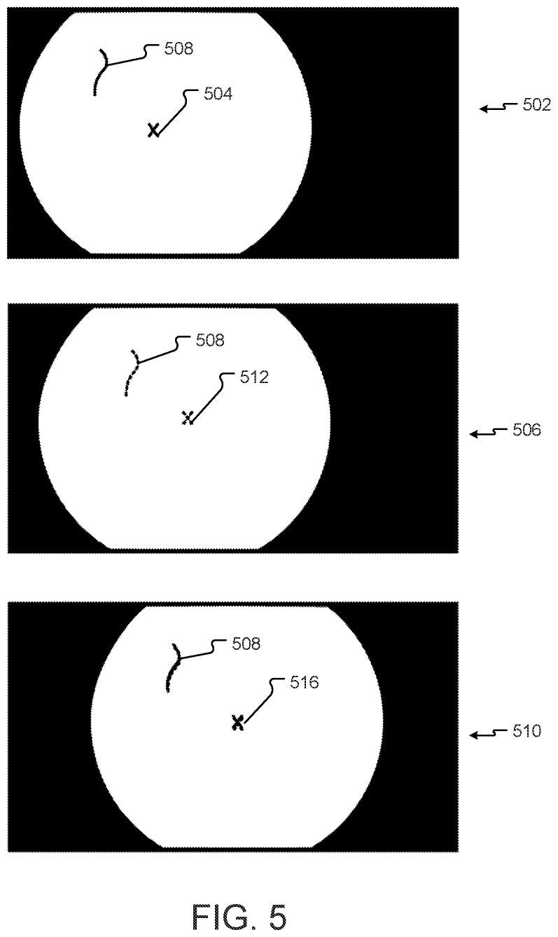

With reference to FIG. 5, aspects of centering channels of two image sensors are shown in accordance with an exemplary embodiment. The movement of the prism assembly 137 to the first orientation or to the second orientation from a default position (where the prism assembly 137 is aligned with the optical axis of the conditioned light 304) may result in a shift in the position upon the image sensor at which images are generated on the first image sensor 332 and/or the second image sensor 344. For instance, and as depicted in FIGS. 3A and 3D, when the prism assembly 137 is in the first orientation, the transmitted light 336 is shifted relative to a centerline 348 of the first image sensor 332, such that the image generated by the first image sensor 332 is shifted (e.g., to the right of the sensor center). Similarly, and as depicted in FIGS. 3B and 3E, when the prism assembly 137 is in the second orientation, the transmitted light 336 is shifted relative to the centerline 348 of the first image sensor 332 in an opposite direction of the shift from the first orientation (e.g., to the left of the sensor center). Such shifting, or “walk-off” may also occur on the second image sensor 344.

The shifting may result in a shift or offset of features of the surgical scene depicted in the images (or series of images such as when a video of the surgical scene is captured) generated by the first image sensor 332 and the second image sensor 344. The shift or offset may make the images or video difficult to view when rendered to the user interface 144 with the offset. To account and/or compensate for the offset, the instructions 128 may be executed to perform feature detection and registration to minimize the offset and/or the images sensors 332, 344 may be physically adjusted relative to the prism assembly 137 to minimize the offset.

In one embodiment, the first image sensor 332 and/or the second image sensor 344 may be positioned on movable stages, such that the first image sensor 332 and/or the second image sensor 344 can be moved in a complementary motion relative to the prism assembly 137 to account for the offset. For example, while in the first orientation the transmitted light 336 from the prism assembly 137 may be shifted a first amount relative to the centerline 348 of the first image sensor 332. In response, the processor 120 may determine an amount of movement of the first image sensor 332 sufficient to center the first image sensor 332 relative to the transmitted light 336. In some embodiments, such information may be known or predetermined (e.g., information stored in the memory 124 and/or the database 156) such that the processor 120 can automatically adjust the position of the first image sensor 332 before, during, or after the prism assembly 137 has been moved into the first orientation. The processor 120 may actuate one or more motors that control the position of the stage or platform to which the first image sensor 332 is attached. A similar adjustment to the position of the second image sensor 344 may also be performed to center the image generated by the second image sensor 344.

Additionally or alternatively, the instructions 128 may, when executed by the processor 120, enable the processor to use one or more feature detection and/or registration techniques known in the art to adjust and center the images generated by the first image sensor 332 and the second image sensor 344. For example, a first image 502 may correspond to an image depicting a feature 508 of a surgical scene captured by the first image sensor 332 when the prism assembly 137 is in the first orientation, such that a center 504 of the first image 502 is offset from a center of the viewing area. Similarly, a second image 506 may correspond to an image depicting the feature 508 of the surgical scene captured by the second image sensor 344 when the prism assembly 137 is in the first orientation. Like the first image 502, a center 512 of the second image 506 may also be offset from the center of the viewing area. The processor 120 may determine the relative shift of the center 504 and the center 512 from the center of the viewing area (e.g., using one or more feature detection algorithms that determine a distance between two points based on pixel values). The processor 120 may also use one or more feature detection algorithms to detect common features (e.g., the feature 508) in both the first image 502 and the second image 506. The processor 120 may then determine an amount of offset between the first image sensor 332 and the second image sensor 344 based on the difference in location of the common features using one or more registration techniques known in the art. Based on the known shifts of each image sensor channel and the difference between the first sensor channel and the second sensor channel, the processor 120 may adjust the depiction of the sensor channels to account of the shifts, resulting in a centered and registered composite image 510 with a center 516 that aligns with the center of the viewing area that can be rendered to, for example, the user interface 144.

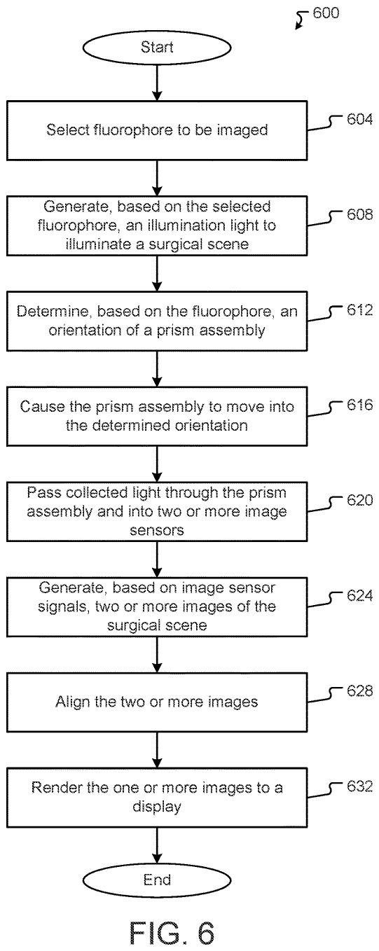

FIG. 6 shows a method 600 according to at least one exemplary embodiment of the present disclosure. The method 600 may be used, for example, to generate one or more images for a selected fluorophore by moving a prism assembly.

The method 600 starts and then proceeds to step 604, where a fluorophore to be imaged is selected. In some embodiments, the fluorophore is chosen manually based on physical input (e.g., the physician selects the fluorophore to be imaged by pressing virtual buttons on a screen rendered on the display 148). In other examples, the imaging mode may be automatically selected based on the current step in the surgery or surgical procedure (e.g., the surgical procedure has proceeded to a step where a fluorescent image of ICG is to be captured).

The method 600 then continues to step 608, wherein a light source comprising a plurality of illuminants generates an illumination light to illuminate a surgical scene, where the illuminants activated are determined based at least partially on the selected fluorophore. The light source may be similar to or the same as the light source 112. The controller 108 may determine, based on the physician input and using instructions and/or algorithms 128, one or more illuminants that should be enabled to generate the required illumination. For example, when a fluorescent image of ICG is to be generated, the controller 108 may determine which illuminants should be illuminated to output an excitation spectrum with a peak wavelength of about 790 nm as well as illuminants required to, if desired, enable the generation a white light image, such as by illuminating red, green and blue illuminants or a broad spectrum white light illumination.

The method 600 then continues to step 612, where the specified orientation of a prism assembly is determined based on the fluorophore. The prism assembly 137 may be moved from a first orientation to a second orientation (or vice versa) based on the selected fluorophore. For instance, when ICG is selected, the processor 120 may determine (based on data stored in the memory 124 and processed by the processor 120) that the prism assembly 137 should be in the first orientation. Alternatively, when Cy5.5 is selected, the processor 120 may determine that the prism assembly 137 should be in the second orientation. In some embodiments, the orientation may be specified by the physician (e.g., via the user interface 144).

The method 600 then continues to step 616, where the prism assembly is moved into the determined orientation. The prism assembly may be moved by the processor 120 based on inputs from the physician (e.g., via the user interface 144) or automatically after executing, for example, instructions 128 stored in the memory 124. The processor 120 may actuate motors coupled with the stage or platform on which the prism assembly 137 is positioned, such that the prism assembly 137 moves from the first orientation to the second orientation (or vice versa).

The method 600 then continues to step 620, where light collected from the surgical scene is captured by the camera head 116 and passed through one or more optical components (e.g., imaging assembly 136) to the prism assembly 137, where the prism assembly 137 splits the light into two or more portions and channels the portions of light to the first image sensor 332 and the second image sensor 344.

The method 600 then continues to step 624, where two or more images of the surgical scene are generated based on image sensor signals. For example, in imaging ICG a true color image may be generated based on the light measured at the first image sensor 332 while a fluorescent image depicting the ICG emission may be generated based on the light measured at the second image sensor 344. The number and type of images generated is in no way limited, and any image discussed herein may be generated in the step 624. The controller 108 may be used along with the instructions 128 to access and implement one or more known image processing algorithms to transform data received from the first image sensor 332 and/or the second image sensor 344 into two or more images.

The method 600 then continues to step 628, where the two or more images are aligned. The processor 120 may process data stored in the memory 124 that enables the processor to implement one or more known image processing algorithms to perform feature detection on the images from the first image sensor 332 and the second image sensor 344 and register the two images. Alternatively or additionally, the processor 120 may recognize, based on the physical properties of the prism assembly 137, previous calibration stored with in the memory 124 of the system 100, etc., the fixed deviation of the images on their respective sensors resulting from moving the prism from a first orientation to a second orientation, and may adjust the position of one or more of the image sensors to compensate for the deviation of one or more of the images due to the movement of the prism from a first orientation to a second orientation. In some embodiments of the method 600, the image sensors may be shifted to perform a gross alignment and image detection may be performed to provide a fine alignment. The processor 120 may then output a composite, centered image of the surgical scene.

The method 600 then continues to step 632, where the two or more images are rendered to a display. The display may be similar to or the same as the display 148. In some embodiments, the two or more images may be captured in the form of a video by repeatedly looping back after step 632 to step 620 and thus repeating steps 620, 624, 628, and 632 for each successive video frame. In such a video display the camera head generally continuously receives light, and images are continuously generated from the sensor measurements. The method 600 then ends. In one embodiment, the composite, centered image of the surgical scene generated in the step 628 is rendered to the display. In some embodiments, the method 600 may then repeat during the course of the surgery or surgical procedure as different fluorophores are selected for imaging, for example, by repeating step 604 and selecting a different fluorophore.

Any of the steps, functions, and operations discussed herein can be performed continuously and automatically.

While the exemplary embodiments illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, sub-combinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving case, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Example aspects of the present disclosure include:

An endoscopic or exoscopic imaging device according to at least one embodiment of the present disclosure comprises: a first image sensor positioned on a first movable stage; a second image sensor positioned on a second movable stage; and a multi-channel prism configured to be moved from a first orientation to a second orientation and configured to separate an input light into: a first spectrally distinct portion of output light directed to the first image sensor, and a second spectrally distinct portion of output light directed to the second image sensor, wherein the multi-channel prism comprises a dichroic filter with a cutoff wavelength that separates the input light into the first and second distinct portions of output light, wherein, when the multi-channel prism is in a first orientation the multi-channel prism receives an incoming image light at a first angle and the cutoff wavelength of the dichroic filter is a first value, wherein, when the multi-channel prism is in a second orientation the multi-channel prism receives the incoming image light at a second angle and the cutoff wavelength of the dichroic filter is a second value different from the first value, and wherein at least one of the first movable stage and the second movable stage is configured to be moved in a complementary motion relative to the multi-channel prism to center images created by at least one of the first image sensor and the second image sensor.

Any of the aspects herein, wherein the first value of the cutoff wavelength of the dichroic filter when the multi-channel prism is in the first orientation is about 720 nanometers (nm).

Any of the aspects herein, wherein the second value of the cutoff wavelength of the dichroic filter when the multi-channel prism is in the second orientation is about 780 nm.

Any of the aspects herein, wherein the multi-channel prism comprises a right angle prism or a pentaprism.

Any of the aspects herein, further comprising: a filter configured to block excitation wavelengths of one or more fluorophores.

Any of the aspects herein, further comprising: a processor; and a memory storing instructions for execution by the processor that, when executed by the processor, enable the processor to: determine an orientation of the multi-channel prism; and cause at least one of the first image sensor and the second image sensor to move relative to the multi-channel prism to compensate for an image offset between the first image sensor and the second image sensor.

Any of the aspects herein, wherein the processor is further enabled to: receive, from the first image sensor, first image data; receive, from the second image sensor, second image data; perform feature detection to determine an offset between the first image sensor and the second image sensor; adjust the first image data to compensate for the offset; and overlay the adjusted first image data and the second image data.

Any of the aspects herein, wherein, when the multi-channel prism is in the first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm.

Any of the aspects herein, wherein, when the multi-channel prism is in the second orientation, the first spectrally distinct portion of output light comprises white light and NIR light with wavelengths between about 700 nm and 780 nm, and second spectrally distinct portion of output light comprises wavelengths greater than about 780 nm.

An imaging system for an endoscope or an exoscope according to at least one embodiment of the present disclosure comprises: a first image sensor positioned on a first movable stage; a second image sensor positioned on a second movable stage; a multi-channel prism configured to be moved from a first orientation to a second orientation and configured to separate, using a dichroic filter with a cutoff wavelength, an input light into a first spectrally distinct portion of output light directed to the first image sensor and a second spectrally distinct portion of output light directed to the second image sensor; a processor; and a memory storing instructions for execution by the processor that, when executed by the processor, enable the processor to: determine an orientation of the multi-channel prism, wherein, when the multi-channel prism is in a first orientation the multi-channel prism receives an incoming image light at a first angle and the cutoff wavelength of the dichroic filter is a first value, and wherein, when the multi-channel prism is in a second orientation the multi-channel prism receives an incoming image light at a second angle and the cutoff wavelength of the dichroic filter is a second value different from the first value; and cause at least one of the first image sensor and the second image sensor to move relative to the multi-channel prism to compensate for an image offset between the first image sensor and the second image sensor.

Any of the aspects herein, wherein the first value of the cutoff wavelength of the dichroic filter when the multi-channel prism is in the first orientation is about 720 nanometers (nm).

Any of the aspects herein, wherein the second value of the cutoff wavelength of the dichroic filter when the multi-channel prism is in the second orientation is about 780 nm.

Any of the aspects herein, wherein the multi-channel prism comprises a right angle prism or a pentaprism.

Any of the aspects herein, further comprising: a filter configured to block excitation wavelengths of one or more fluorophores.

Any of the aspects herein, wherein, when the multi-channel prism is in the first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm.

Any of the aspects herein, wherein, when the multi-channel prism is in the second orientation, the first spectrally distinct portion of output light comprises white light and NIR light with wavelengths between about 700 nm and 780 nm, and second spectrally distinct portion of output light comprises wavelengths greater than about 780 nm.

An imaging system for an endoscope or an exoscope according to at least one embodiment of the present disclosure comprises: a first image sensor; a second image sensor; a multi-channel prism configured to be moved from a first orientation to a second orientation and configured to separate, using a dichroic filter with a cutoff wavelength, an input light into a first spectrally distinct portion of output light directed to the first image sensor and a second spectrally distinct portion of output light directed to the second image sensor, wherein, when the multi-channel prism is in a first orientation the multi-channel prism receives an incoming image light at a first angle and the cutoff wavelength of the dichroic filter is a first value, and wherein, when the multi-channel prism is in a second orientation the multi-channel prism receives an incoming image light at a second angle and the cutoff wavelength of the dichroic filter is a second value different from the first value; a processor; and a memory storing instructions for execution by the processor that, when executed by the processor, enable the processor to: receive, from the first image sensor, first image data; receive, from the second image sensor, second image data; perform feature detection to determine an offset between the first image sensor and the second image sensor; adjust the first image data to compensate for the offset; and overlay the adjusted first image data and the second image data.

Any of the aspects herein, wherein the multi-channel prism comprises a right angle prism or a pentaprism.

Any of the aspects herein, further comprising: a filter configured to block excitation wavelengths of one or more fluorophores.

Any of the aspects herein, wherein, when the multi-channel prism is in a first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm, and wherein, when the multi-channel prism is in a second orientation, the first spectrally distinct portion of output light comprises white light and NIR light with wavelengths between about 700 nm and 780 nm, and second spectrally distinct portion of output light comprises wavelengths greater than about 780 nm.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. An endoscopic or exoscopic imaging device, comprising:
a first image sensor positioned on a first movable stage;
a second image sensor positioned on a second movable stage; and
a multi-channel prism configured to be moved from a first orientation to a second orientation and configured to separate an input light into:
a first spectrally distinct portion of output light directed to the first image sensor, and
a second spectrally distinct portion of output light directed to the second image sensor,
wherein the multi-channel prism comprises a dichroic filter with a cutoff wavelength that separates the input light into the first and second distinct portions of output light,
wherein, when the multi-channel prism is in a first orientation the multi-channel prism receives an incoming image light at a first angle and the cutoff wavelength of the dichroic filter is a first value, wherein, when the multi-channel prism is in a second orientation the multi-channel prism receives the incoming image light at a second angle and the cutoff wavelength of the dichroic filter is a second value different from the first value, and wherein at least one of the first movable stage and the second movable stage is configured to be moved in a complementary motion relative to the multi-channel prism to center images created by at least one of the first image sensor and the second image sensor.

2. The imaging device of claim 1, wherein the first value of the cutoff wavelength of the dichroic filter when the multi-channel prism is in the first orientation is about 720 nanometers (nm).

3. The imaging device of claim 2, wherein the second value of the cutoff wavelength of the dichroic filter when the multi-channel prism is in the second orientation is about 780 nm.

4. The imaging device of claim 1, wherein the multi-channel prism comprises a right angle prism or a pentaprism.

5. The imaging device of claim 1, further comprising:
a filter configured to block excitation wavelengths of one or more fluorophores.

6. The imaging device of claim 1, further comprising:
a processor; and
a memory storing instructions for execution by the processor that, when executed by the processor, enable the processor to:
determine an orientation of the multi-channel prism; and
cause at least one of the first image sensor and the second image sensor to move relative to the multi-channel prism to compensate for an image offset between the first image sensor and the second image sensor.

7. The imaging device of claim 6, wherein the processor is further enabled to:
receive, from the first image sensor, first image data;
receive, from the second image sensor, second image data;
perform feature detection to determine an offset between the first image sensor and the second image sensor;
adjust the first image data to compensate for the offset; and
overlay the adjusted first image data and the second image data.

8. The imaging device of claim 1, wherein, when the multi-channel prism is in the first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm.

9. The imaging device of claim 8, wherein, when the multi-channel prism is in the second orientation, the first spectrally distinct portion of output light comprises white light and NIR light with wavelengths between about 700 nm and 780 nm, and second spectrally distinct portion of output light comprises wavelengths greater than about 780 nm.

10. An imaging system for an endoscope or an exoscope, comprising:
a first image sensor positioned on a first movable stage;
a second image sensor positioned on a second movable stage;
a multi-channel prism configured to be moved from a first orientation to a second orientation and configured to separate, using a dichroic filter with a cutoff wavelength, an input light into a first spectrally distinct portion of output light directed to the first image sensor and a second spectrally distinct portion of output light directed to the second image sensor;
a processor; and
a memory storing instructions for execution by the processor that, when executed by the processor, enable the processor to:
determine an orientation of the multi-channel prism, wherein, when the multi-channel prism is in a first orientation the multi-channel prism receives an incoming image light at a first angle and the cutoff wavelength of the dichroic filter is a first value, and wherein, when the multi-channel prism is in a second orientation the multi-channel prism receives an incoming image light at a second angle and the cutoff wavelength of the dichroic filter is a second value different from the first value; and
cause at least one of the first image sensor and the second image sensor to move relative to the multi-channel prism to compensate for an image offset between the first image sensor and the second image sensor.

11. The imaging system of claim 10, wherein the first value of the cutoff wavelength of the dichroic filter when the multi-channel prism is in the first orientation is about 720 nanometers (nm).

12. The imaging system of claim 11, wherein the second value of the cutoff wavelength of the dichroic filter when the multi-channel prism is in the second orientation is about 780 nm.

13. The imaging system of claim 10, wherein the multi-channel prism comprises a right angle prism or a pentaprism.

14. The imaging system of claim 10, further comprising:
a filter configured to block excitation wavelengths of one or more fluorophores.

15. The imaging system of claim 10, wherein, when the multi-channel prism is in the first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm.

16. The imaging system of claim 15, wherein, when the multi-channel prism is in the second orientation, the first spectrally distinct portion of output light comprises white light and NIR light with wavelengths between about 700 nm and 780 nm, and second spectrally distinct portion of output light comprises wavelengths greater than about 780 nm.

17. An imaging system for an endoscope or an exoscope, comprising:
a first image sensor;
a second image sensor;
a multi-channel prism configured to be moved from a first orientation to a second orientation and configured to separate, using a dichroic filter with a cutoff wavelength, an input light into a first spectrally distinct portion of output light directed to the first image sensor and a second spectrally distinct portion of output light directed to the second image sensor, wherein, when the multi-channel prism is in a first orientation the multi-channel prism receives an incoming image light at a first angle and the cutoff wavelength of the dichroic filter is a first value, and wherein, when the multi-channel prism is in a second orientation the multi-channel prism receives an incoming image light at a second angle and the cutoff wavelength of the dichroic filter is a second value different from the first value;
a processor; and
a memory storing instructions for execution by the processor that, when executed by the processor, enable the processor to:
receive, from the first image sensor, first image data;
receive, from the second image sensor, second image data;
perform feature detection to determine an offset between the first image sensor and the second image sensor;
adjust the first image data to compensate for the offset; and
overlay the adjusted first image data and the second image data.

18. The imaging system of claim 17, wherein the multi-channel prism comprises a right angle prism or a pentaprism.

19. The imaging system of claim 17, further comprising:
a filter configured to block excitation wavelengths of one or more fluorophores.

20. The imaging system of claim 17, wherein, when the multi-channel prism is in a first orientation, the first spectrally distinct portion of output light comprises white light and near infrared (NIR) light with wavelengths between about 700 nanometers (nm) and 720 nm, and the second spectrally distinct portion of output light comprises wavelengths greater than about 720 nm, and wherein, when the multi-channel prism is in a second orientation, the first spectrally distinct portion of output light comprises white light and NIR light with wavelengths between about 700 nm and 780 nm, and second spectrally distinct portion of output light comprises wavelengths greater than about 780 nm.
